(19) United States
(12) Patent Application Publication
(10) Pub. No.: US 2003/0180235 A1
(43) Pub. Date: Sep. 25, 2003

(54) Matrix for preparing microparticles or nanoparticles, method for making said particles and resulting particles

(75) Inventors: Philippe Grisoni, Bey Sur Seille (FR); Marc Pauly, Chateau-Salins (FR)

Correspondence Address: YOUNG & THOMPSON, 745 SOUTH 23RD STREET 2ND FLOOR, ARLINGTON, VA, 22202

(73) Assignee: LABORATOIRES SEROBIOLOGIQUES, PULNOY (FR)

(21) Appl. No.: 10/388,658

(22) Filed: Mar. 17, 2003

Related U.S. Application Data
(63) Continuation of application No. 09/623,128, filed on Oct. 10, 2000.
Application No. 09/623,128 is a 371 of international application No. PCT/FR99/00385, filed on Feb. 19, 1999.

(30) Foreign Application Priority Data
Feb. 27, 1998 (FR) 98 02578

(51) Int. Cl. A61K 7/42; A61K 38/43; A61K 9/16; A61K 9/50; B01J 13/02; B01J 13/04

(52) U.S. Cl. 424/59; 424/490; 264/4.1; 424/94.1

(57) ABSTRACT

A matrix for preparing microparticles or nanoparticles, method for making the particles and resulting particles, more particularly a matrix for forming microparticles or nanoparticles by hot dispersion in a non-miscible gas or liquid medium, characterised in that it consists of at least a solid hydrophobic element at room temperature and of at least a liquid element, hydrophobic and/or lipophilic, hot mixed by solubilizing the solid element(s) in the liquid element(s), the matrix having a melting point ranging between 25° C. and 85° C.

The present invention relates to the field of encapsulation and vectorization of substances, agents or ingredients having specific properties, particularly in the field of cosmetics, and has for its object a hydrophobic or lipidic matrix for the preparation of microparticles and/or of nanoparticles, a process for the production of such particles and the particles thus obtained.

### BACKGROUND OF THE INVENTION

Dispersed systems based on lipids have been studied for a long time in the cosmetic industry with the object of encapsulating and vehiculing the active principles in the form of microparticles or nanoparticles (liposomes, niosomes).

Several methods for the preparation of lipidic solid nanoparticles and microparticles using the technique of thermofusion (or “hot-melt”) have been described and used.

According to this technique, a melted wax is dispersed with agitation in water or an immiscible liquid brought to the temperature of the melted wax. The dispersion obtained is then cooled to permit the solidification of the droplets of cast wax.

### DESCRIPTION OF THE RELATED ART

Thus, the French patent application published under the number 2737668 describes a process for the production of balls from lipidic substances constituted by a wax or a mixture of waxes, with a melting point between 25° C. and 60° C., obtained after dispersion with agitation and then cooling, in the presence of surface active agents, of melted waxes, in an aqueous phase (at the temperature of the melted waxes) comprising water and a hydrophilic polymer.

Patent applications PCT No. WO 93/05768 and No. WO 94/20072 describe a process for obtaining solid lipidic nanoparticles after homogenization under high pressure of a hot aqueous dispersion of a wax or a mixture of melted waxes, if desired in the presence of surface active agents, the nanoparticles solidifying upon cooling.

However, a principal drawback of the micro- and nanoparticle arrangements described above is their limited power to encapsulate liquid lipophilic active principles.

Thus, the liposomes and niosomes used have a weak capacity for trapping such ingredients in their lipidic structure. Similarly, the solid lipidic microparticles and nanoparticles produced cannot incorporate large quantities of liquid active principles, without the risk of modification of their structure and hence without risk of alteration of their stability.

### SUMMARY OF THE INVENTION

The present invention has particularly for its object to overcome the mentioned drawbacks and to provide a technical solution for the preparation of heavily loaded microparticles and/or nanoparticles, in particular with liquid active principles.

To this end, the present invention has for its object a matrix adapted to form microparticles or nanoparticles by hot dispersion in an immiscible gaseous or liquid medium, characterized in that it is comprised of at least one hydrophobic body solid at ambient temperature and at least one liquid body, hydrophobic and/or lipophilic, mixed hot by solubilization of the solid body or bodies in the liquid body or bodies, said matrix having a melting temperature comprised between 25° C. and 85° C.

According to a first characteristic of the invention, the solid body or mixture of solid bodies and, as the case may be, the liquid body or mixture of liquid bodies, as well as if desired their proportion, is or are selected such that the melting temperature of the resulting matrix is comprised between 50° C. and 75°.

This matrix can be used as such or, preferably, added to one or several active principles of hydrophobic, hydrophilic or lipophilic nature.

The solid hydrophobic body, or the mixture of solid hydrophobic bodies, constitutes 10 to 90%, preferably 30 to 60% by weight, of the matrix and is selected from the group comprised by solid fatty bodies, waxes and hydrophobic polymers used in cosmetic compositions, preferably of the type having melting temperatures comprised between 25° and 150° C.

Thus the solid hydrophobic body, or the mixture of solid hydrophobic bodies, can particularly be selected for example from the following substances or compounds:

long chain fatty acid mono-, di- and triglycerides such as products sold under the names Dynasan, Softisan, Witepsol or Novata, for example;

lipophilic solid active principles in general;

polyol esters such as glycol stearate, for example;

hydrogenated vegetable oils such as hydrogenated castor oil, hydrogenated jojoba oil or else hydrogenated coconut oils, for example;

fatty acids such as stearin, their esters such as cetyl palmitate, myristyl myristate, for example;

fatty alcohols such as cetyl alcohol or behenic alcohol, for example;

natural waxes such as beeswax, carnauba wax, candellila wax, karite butter or illipe butter, for example;

synthetic waxes such as silicone waxes sold under the name “Abil Wax” or ketones such as palmitone and stearone, for example;

fatty acids or esters of lanolin, or other derivatives of solid lanolin;

synthetic hydrophobic polymers such as polyethylene or polypropylene, for example;

sterols such as soya sterol and cholesterol, for example;

waxes derived from petroleum such as for example paraffin, ozokerite or microcrystalline waxes;

gums and resins of natural origin such as colophane, lac gum or sandarac gum, for example.

The liquid hydrophobic body, or the mixture of liquid hydrophobic bodies, constitutes from 10 to 90%, preferably 40 to 70% by weight of the matrix and is selected from solvents, particularly hot, for the solid hydrophobic body or bodies entering into the composition of the matrix, in particular liquid oils and liquid liposoluble active principles used in cosmetic compositions.

Thus, the liquid hydrophobic body, or the mixture of liquid hydrophobic bodies, could particularly be selected, for example, from the following substances or compounds:

vegetable oil such as jojoba oil, camelia oil, macadamia oil, sweet almond, sesame oil or oil of tournesol, for example;

gylcerides, and more particularly short chain or unsaturated fatty acid glycerides, such as glycerol oleate or Miglyol 812, for example;

liquid esters of polyols, such as Miglyol 840, for example;

liquid fatty acid esters such as isopropyl myristate, for example;

liquid branched and unsaturated fatty acids, such as isosteric acid and oleic acid, for example;

short chain branched fatty acids, such as for example isododecanol sold under the name Eutanol G;

ethers, such as dioctyl ether for example;

paraffin oils and isoparafins;

silicone oils, cyclic, alkylated or phenolated, dimethiconols or dimethicones, for example;

synthetic oils such as isohexadecane, dioctyl cyclohexane, perhydrosqualene and halogenated oils, for example;

polyisobutylene and hydrogenated polybutylene.

However, the liquid hydrophobic body, or the equivalent mixture, could be totally or partially replaced by a liquid lipophilic active principle.

In this modified embodiment of the invention, the matrix comprises, in addition to the liquid hydrophobic body, or in replacement of this latter, between 10 and 90%, preferably between 40 and 70% by weight of a liquid lipophilic body or a mixture of liquid lipophilic bodies, in the form of one or several liquid lipophilic active principles.

The present invention also has for its object a process for the production of microparticles or nanoparticles, particularly adapted to encapsulate and/or to vectorize one or more active substances, in particular lipophilic, hydrophobic or hydrophilic, characterized in that it consists in preparing a matrix by solubilizing with heating at least one solid hydrophobic body, at least at ambient temperature, in at least one liquid body which is hydrophobic and/or hydrophilic, at a temperature higher than the melting temperature of the resulting matrix, in dispersing this hot matrix in liquid or substantially liquid form, in an immiscible gaseous or liquid medium and cooling after dispersion or in the course of dispersion, the resulting matrix for its solidification in the form of microparticles or nanoparticles.

Preferably, the melting temperature of the resulting matrix is comprised between 25° C. and 85° C., preferably between 50° C. and 75° C.

This matrix will preferably have a composition of solid hydrophobic bodies and liquid hydrophobic bodies as described above, said liquid hydrophobic body or bodies being if desired partially or totally substituted with one or more liquid lipophilic active principles as indicated above.

As to the processes for dispersion of this matrix for obtaining the desired microparticles or nanoparticles, it is possible to use various techniques known to those in the art such as for example emulsification, atomization or stalagmopoiesis.

According to a first embodiment of the invention, the matrix can be dispersed in liquid or in substantially liquid state, as the case may be with added surface-active substances, in an immiscible liquid medium, comprising if desired surface active substances and having a temperature of substantially the same value as that of said matrix and then cooling the mixture sufficiently to solidify the hydrophobic matrix disposed in the medium and encapsulating the active substance or substances.

In this case, the medium immiscible with the lipidic matrix could be selected from:

water;

liquids immiscible with oils, such as glycerin and propylene glycol, for example, or a dimethicone oil;

water with hydrosoluble additives added such as humidifying agents, preservatives, cryoprotective agents, active principles, pH adjusters or the like;

water with the addition of one or several hydrophilic, ionic and/or non-ionic polymers, stabilizing interfacial and/or viscosity agents such as for example proteins, polysaccharides (xanthane, carragheenin, galactomanane, alginate, modified starches . . . ), cellulosic derivatives (methyl cellulose, hydroxyethylcellulose . . . ), acrylic polymers and copolymers (for example those known by the name Carbopol, Pemulen or Acrysol), polyacrylamide or cationic polymers derived from polyvinylpyrrolidone, from guar gum or from hydroxyethylcellulose, for example;

water with added dispersible stabilizing agents such as silica, aluminum silicate and magnesium silicate (as called Veegum) or the like.

As a function of the means used to produce the dispersions (emulsification with helical and/or shearing turbine agitation, sonication, homogenization under high pressure), a matrix of identical composition could lead to two forms of encapsulization and vectorization.

For the preparation of nanoparticles of a size less than 500 nanometers, the presence of surface active agents is generally necessary. In this case, they are selected from non-ionic and/or ionic surface active agents now used in cosmetics and called for example in the second edition of the “Encyclopedia of Surfactants” published by the review “Cosmetics and Toiletries”, and preferably, from surface active agents of natural origin such as for example phospholipids and derivatives, aminated acid derivatives such as acylglutamates, esters of sucrose, alkylglucosides, distearoyl ethyl hydroxy ethyl methosulfate (known by the commercial name “Dehyquart F75”) or auto-emulsifying bases of vegetable origin known under the commercial name “Tegin V”, this or these surface active agents can be incorporated in one or the other of the phases as a function of their physico-chemical properties. The surface active agent can be formed in situ in the case of saponification of a fatty acid present in the matrix as a solid hydrophobic body.

According to a second embodiment of the invention, it can be provided to disperse the hot matrix in liquid phase or substantially liquid phase in a gaseous or liquid medium, when cold, so as to obtain by spontaneous solidification fine liquid droplets dispersed in the form of solid microparticles.

Thus, the hot lipidic solution constituting the matrix can, for example, be dispersed in the form of fine liquid droplets in a cold air chamber in which they solidify spontaneously to form microparticles of small size.

Microparticles of a size substantially comprised between 0.5 and 5 millimeters can also be obtained by the technique known as stalagmopoiese, which consists in solidifying droplets of hot lipidic solution after their free fall into a cold air chamber or countercurrent to cold water, or in a cold immiscible liquid.

By way of example of hydrophobic active principles that can be integrated into and carried by the particles obtained by the process according to the invention incorporated in said microparticles or nanoparticles during their formation because of their solubilization or their dispersion in the liquid phase matrix, can be cited:

liposoluble vitamins of the acetate, linoleate, tocopherol nicotinate, retinol palmitate, vitamin A propionate type,

ceramids of various origins, for example, biological, synthetic, biotechnological or vegetable (soya, tournesol, wheat . . . etc.),

sterols,

α bisabolol,

phytosterols,

lecithins, phospholipids,

liposoluble colorants and pigments,

solar filters such as diethylhexyl paramethoxycinnamate,

essential oils and perfumes.

The hydrophilic active principles, particularly solid and carried in an adsorbed form, could for example be selected from:

dihydroxyacetone;

ascorbyl magnesium phosphate (vitamin C);

enzymes;

proteins, protein hydrolysates and aminated acid derivatives;

polysaccharides;

hydrosoluble solar filters;

salicylic acid;

vitamin C or D derivatives.

The hydrophobic, lipophilic and/or hydrophilic active substances to be, as the case may be, encapsulated or vectorized, are incorporated for example by dispersion or solubilization in the matrix in the liquid phase before dispersion and cooling of this latter.

So as to produce systems with active substances with double vectorization, there can moreover be provided a step consisting in adsorbing hydrophilic active substances present in aqueous phase on the surface of formed microparticles or nanoparticles imprisoning a lipophilic active principle

Carrying out the above step is facilitated when the process of production according to the invention integrates two successive operative phases consisting in producing first positively or negatively charged microparticles or nanoparticles, by selecting the natures and proportions of the constituents of the matrix and of the dispersion medium and by adjusting the pH of said dispersion medium in a suitable manner, and then complexing said microparticles or nanoparticles with one or several active principles of opposite charge or having local opposite charges in their structure.

Thus, this phenomenon of adsorption can be amplified by producing positively or negatively charged nanoparticles and/or microparticles, complexed with an active principle of opposite charge or having both in its structure.

Thus, by adjusting the nature and proportions of the reaction products used, as well as the pH of the medium, numerous hydrophilic substances such as proteins, enzymes, protein hydrolysates or amino acids are capable of being fixed and immobilized on the surface of negatively or positively charged nanoparticles and/or microparticles, anionic active principles, such as for example magnesium ascorbyl phosphate or solar filters with a sulfonate group adsorbing preferentially on positively charged particles. The complexing has the effect of rupturing the formed interface, to give rise to microparticulate aggregates comprised of nanoparticles and/or microparticles agglomerated by a suitable complexing agent.

At this stage it can also be provided to isolate from the dispersion medium the microparticulate aggregates formed by adsorption of charged hydrophilic active principles by ionic complexing on microparticles or nanoparticles of opposite charge, and then drying said microparticulate aggregates, for example by lyophilization or by drying in a fluidized bed, to obtain hydrophobic powders insoluble in water with a double activity potential.

The microparticles or nanoparticles in suspension obtained by the process according to the invention can also be dehydrated so as to lead to a final pulverulent product.

The present invention also has for its object microparticles or nanoparticles obtained particularly by means of the process of production described above and having sizes comprised between 5 millimeters and 10 nanometers, preferably between 50 nanometers and 3 millimeters, characterized in that they are formed from a hydrophobic or lipophilic matrix such as set forth above.

The production process described above permits, accordingly, preparing:

either nanoparticles and/or microparticles carrying hydrophobic active principles,

or nanoparticles and/or microparticles carrying hydrophilic active principles, in particular microcrystalline solid hydrophilic active principles,

or nanoparticles and/or microparticles carrying hydrophobic active principles included in the matrix itself, and hydrophilic active principles (in particular solid hydrophilic active principles) encapsulated in the hydrophobic matrix or adsorbed on the surface of the latter.

These nanoparticles or microparticles could particularly enclose substances selected from the group formed by liposoluble or lipodispersable active principles, liposoluble colorants, hydrophilic active principles in dispersed form, organic and mineral fillers such as for example pigments, nacrous agents or talc, maintained in suspension by stabilizing additives such as bentones, bentone derivatives, silica or any other analogous ingredient capable of stabilizing the oily suspensions.

Moreover, as already mentioned above, these particles could also carry one or several hydrophobic active principles incorporated in said microparticles or nanoparticles during their formation because of their solubilization or their dispersion in the matrix in liquid phase, selected from the group indicated above, or else carry hydrophilic active principles in adsorbed form.

Because of the high melting point of the hydrophobic matrix, the obtained systems, no matter whether simple nanoparticles and/or simple microparticles, nanoparticles and/or microparticles at the surface of which are adsorbed various molecules or else nanoparticles and/or microparticles complexed in the form of aggregates, have a high hot stability and can therefore be subjected successively to stability tests currently carried out in the cosmetic industry.

It should be noted that no chemical reaction, particularly of polymerization, is necessary to lead to the formation of microparticles or nanoparticles in the scope of the matrix according to the invention. The waxes used play the structural and stabilizing role for said micro- or nanoparticles and the solvent or solvents of the waxes (liquid oils) are used as crystallization supports and do not undergo any chemical transformation in the scope of the process according to the invention.

Finally, the invention also relates to a cosmetic and/or dermopharmaceutical composition for the care of the skin and/or nails, characterized in that it comprises as active ingredient or a vehicle for active ingredients, microparticles and/or nanoparticles as described above.

These microparticles and/or nanoparticles produced with the matrix according to the invention, could desirably be incorporated in various types of cosmetic and/or dermopharmaceutical products for the care of the skin and the nails, in forms customarily used.

In particular, they will be used in the framework of oil emulsions in water, aqueous gels and lotions, foaming cleaning products for the skin and nails, of the gel douche type, bath foam, shampoo or the like, makeup products, loose or compact powders, mascaras, eye pencils, permanent wave or coloring products for hair. The dosage of utilization of these nanoparticles and/or microparticles could vary as a function of the nature of the active principle of these nanoparticles or microparticles, and the concentration of the desired active principle in the final product, from 0.1 to 20%, preferably 0.5 to 10% by weight of the cosmetic and/or dermopharmaceutical composition.

By way of illustrative and non-limiting examples of the possibilities of application of the invention, there will be given hereafter the description of several examples of practical embodiment.

### EXAMPLE 1

Composition of a lipidic matrix for the preparation of nanoparticles or microparticles.

| Chemical name | Commercial name | % by weight |
|---|---|---|
| Behenic alcohol | Lanette 22 | 30 |
| Hydrogenated caster oil | Cutina HR | 10 |
| Octyl stearate | Cetiol 868 | 60 |

The above composition is prepared by carrying out successively the following operations.

The three components are brought to a temperature of 90° C., so as to obtain a homogeneous clear solution.

The solution is then cooled and maintained in a thermostatic bath at a temperature of 75° C.

The drop point of the solidified mixture is 65.7° C., measured by a Mettler PF5-PF53 apparatus.

### EXAMPLE 2

Composition of a lipidic matrix for the preparation of nanoparticles, of microparticles.

| | Commercial | |
|---|---|---|
| Chemical name | name | weight |
| Carnauba wax | / | 20.00 g |
| Tristearine | Dynasan 118 | 70.00 g |
| Hydrogenated coconut glycerides | Witepsol E 85 | 10.00 g |
| Capric/caprylic triglyceride | Miglyol 812 | 100.00 g |

The above composition is prepared by carrying out successively the following operations.

The three first components are brought to a temperature of 90° C. so as to obtain a homogeneous and clear solution. The caprylic/capric triglyceride is heated to 45° C. then added with agitation to the first phase.

The drop point of the solidified mixture is 65.7° C., measured by a Mettler PFS-PF53 apparatus.

### EXAMPLE 3

Preparation of microparticles of 250 microns

| | | |
|---|---|---|
| A | Lipidic matrix of example 2 | 200.00 g |
| B | Osmolized water | 998.00 g |
| | Guar Hydroxypropyltrimonium (Jaguar C13S) | 2.00 g |
| | Preservative | qs |

Operative Mode of Preparation:

Phase A at 75° C. is dispersed with a helical agitation at 200 rpm in the B phase at 75° C., then the dispersion is cooled with agitation in an ice bath to permit solidification of microparticles.

The microparticles are recovered on a sieve and rinsed with osmosed water.

They can be dried and incorporated in various cosmetic formulations as emulsions and hydrogels.

### EXAMPLE 4

Preparation of Microparticles of 40 microns

| | | |
|---|---|---|
| A | Lipidic matrix of Example 1 | 400.0 g |
| B | Osmosed water | 1976.0 g |
| | Carbomer | 4.0 g |
| C | 1N NaOH | 20.0 g |

Operating Mode of Preparation:

Phase A at 75° C. is dispersed with turbine agitation of the Polytron type at 2500 rpm for 5 minutes in phase B at 75° C. in a 5 liter Pyrex beaker in a thermostatic bath.

The dispersion is then cooled with agitation in an ice water bath, phase C being introduced at 40° C., the cooling being continued until ambient temperature.

The microparticles are maintained in suspension in their production medium and can be incorporated in emulsion and cosmetic hydrogels.

### EXAMPLE 5

Preparation of nonionic nanoparticles

| | | |
|---|---|---|
| A | Lipidic matrix of Example 1 | 15.00% |
| | Sisterna SP 10 C (sucrose ester) | 0.95% |
| B | Osmosed water | qsp 100 |
| | Sisterna SP 70 C (sucrose ester) | 3.25% |
| | Preservative | qs |

Operative Mode of Preparation:

Phase A at 75° C. is dispersed in phase B at 75° C. with Polytron turbine agitation, 2500 rpm.

The obtained emulsion is homogenized under 800 bars of pressure, three homogenized stations being carried out successively.

The obtained suspension is cooled with moderate agitation.

The mean size of the nanoparticles measured by spectroscopy with proton correlation is 108 nm.

### EXAMPLE 6

Preparation of Microparticles of 200 Microns from Primary Material of Vegetable Origin

| | | |
|---|---|---|
| A | Carnauba wax | 18.00 g |
| | Candellia wax | 50.00 g |
| | Sphingoceryl Veg (trademark) | 130.00 g |
| | Anhydrochrome oxide | 2.00 |
| B | Osmosed water | 998.00 g |
| | Guar hydroxypropyltrimonium | 2.00 g |
| | (Jaguar C13S) | |
| | Preservative | qs |

Operative Mode of Preparation:

The waxes are melted at 90° C., the chromium oxide is dispersed in Sphingoceryl VEG at 40° C. and the dispersion is then added to the melted waxes. Phase A is maintained at 75° C. and then dispersed in phase B at 75° C. with helical agitation at 800 rpm. The obtained suspension is cooled with agitation to ambient temperature. The microparticles of green color isolated after screening can be incorporated in a hydrogel or an O/W emulsion.

### EXAMPLE 7

Preparation of Exfoliant Microparticles of 500 Microns

| | | |
|---|---|---|
| A | Behenic alcohol | 92.00 g |
| | Hydrogenated caster oil | 8.00 g |
| | Cetearyl isononanoate | 20.00 g |
| | Tocopherol nicotinate | 80.00 g |
| B | Osmosed water | 997.00 g |
| | Carbomer | 0.50 g |
| | 1N NaOH | 2.50 g |
| | Preservative | qs |

Phase A at 75° C. is dispersed in phase B at 75° C. with helical agitation at 400 rpm.

The dispersion is cooled to ambient temperature, the microparticles are isolated by screening and can be incorporated in an exfoliant massage gel, an active thinning principle being liberated in proportion to the application.

### EXAMPLE 8

Preparation of Microparticles of 40 Microns Enriched in Vitamin E

| | | |
|---|---|---|
| A | Behenic alcohol | 65.00 g |
| | Hydrogenated caster oil | 30.00 g |
| | Tocopherol acetate | 100.00 g |
| B | Osmosed water | 988.00 g |
| | Carbomer | 2.00 g |
| | 1N NaOH | 10.00 g |
| | Preservative | qs |

Phase A at 75° C. is dispersed in phase B at 75° C. with Polytron turbine agitation at 2500 rpm.

The dispersion is cooled to ambient temperature. The microparticles are maintained in suspension in the preparation medium.

### EXAMPLE 9

Preparation of Nanoparticles Enriched in Vitamin E

| | | |
|---|---|---|
| A | Behenic alcohol (Lanette 22) | 6.50% |
| | Hydrogenated caster oil (Cutina HR) | 1.50% |
| | Tocopherol acetate | 10.00% |
| B | Osmosed water | qsp 100 |
| | Soy sterol 25 OE (General E 25) | 4.00% |
| | Preservative | qs |

Operative Mode of Preparation:

Phase A at 75° C. is dispersed in phase B at 75° C. with Polytron type turbine agitation at 2500 rpm. The obtained emulsion is homogenized under 800 bars of pressure at 75° C., two homogenizations being carried out. The obtained suspension is cooled with moderate agitation.

The mean size of the nanoparticles, measured by spectroscopy with photon correlation, is 110 nm.

### EXAMPLE 10

Preparation of Microparticles of 150 Microns Enriched in Photoprotective Agents

| | | |
|---|---|---|
| A | Carnauba wax | 17.00 g |
| | Candellila wax | 27.00 g |
| | Behenic alcohol | 20.00 g |
| | Sesame oil | 10.00 g |
| | Hydrophobic titanium dioxide | 20.00 g |
| | Parsol MCX | 80.00 g |
| | Parsol 1789 | 20.00 g |
| B | Osmosed water | 998.00 g |
| | Guar hydroxypropyltrimonium | 2.00 g |
| | (Jaguar C13S) | |
| | Preservative | qs |

Operative Mode of Preparation:

Phase A is melted at 75° C., the titanium dioxide is dispersed with turbine agitation. Phase A is dispersed with helical agitation at 900 rpm in phase B at 75° C. The dispersion is cooled to ambient temperature. The microparticles are isolated by screening and can be incorporated in hydrogels and/or solar emulsions.

### EXAMPLE 11

Preparation of Positively Charged Nanoparticles

| | | |
|---|---|---|
| A | Dehyquart F75 | 2.50% |
| | Lanette 22 | 2.00% |
| | Cutina HR | 0.30% |
| | Jojoba oil | 4.50% |
| | Karita butter | 0.70% |
| B | Osmosed oil | qsp 100 |
| | Preservative | qs |

Operative Mode of Preparation:

Phase A at 75° C. is dispersed and phase B at 75° C. with Polytron type turbine agitation at 2500 rpm. The obtained emulsion is homogenized under 800 bars pressure, three homogenizations being carried out. The obtained suspension is cooled with moderate agitation.

The mean size of the nanoparticles, measured by spectroscopy with photon correlation, is 174 nm.

### EXAMPLE 12

Complexing of Nanoparticles

| | | |
|---|---|---|
| A | Cationic nanoparticles of Example 11 | 30.00% |
| | Osmosed water | 40.00% |
| B | Wheat protein hydrolysate (Vegeles WP) | 5.00% |
| | Osmosed water | 25.00% |

Operative Mode of Preparation:

Phases A and B are mixed with moderate agitation at ambient temperature.

The microparticulate aggregates formed can be separated by filtration, or the obtained dispersion can be dried by the drying technique by pulverization to give lipidic microparticles complexed with a protein derivative.

The microparticles thus obtained can be incorporated in products for capillary care (hydratants, bioconditioners).

### EXAMPLE 13

Preparation of Microparticles of 1.5 Micron in the Form of a Final Product

| | | |
|---|---|---|
| A | Lipidic matrix of Example 1 | 10.00% |
| B | Osmosed water | qsp 100 |
| | Carbomer | 0.40% |
| | Vegeseryl HGP (soya protein) | 15.00% |
| | Preservative | qs |
| C | 1N NaOH | 2.00% |

Operative Mode of Preparation:

Phase A at 75° C. is dispersed with Polytron turbine agitation at 2500 rpm in phase B at 75° C.

The obtained dispersion is homogenized under a pressure of 500 bars at 75° C., then cooled with moderate agitation, phase C being added at 40° C.

The dispersion is cooled to ambient temperature and is in the form of a fluid serum liberating a soy protein and its matrix upon application.

The skin is covered with an imperceptible fatty film-resistant water.

### EXAMPLE 14

Cosmetic product for face care in the form of a cream, comprising nanoparticles enriched in Vitamin E

| | | |
|---|---|---|
| A | Sorbitan palmitate | 3.50% |
| | Glycerol stearate | 1.50% |
| | Cetyl alcohol | 2.50% |
| | Cetearyl Isononanoate | 7.00% |
| | Paraffin oil | 3.00% |
| | Octyldodecanol | 5.50% |
| | Dimethicone | 2.00% |
| | Preservative | 0.30% |
| B | Water | 64.10% |
| | Preservative | 0.40% |
| | Glycerin | 4.00% |
| | Sodium cetearyl sulfate | 1.20% |
| C | Nanoparticles according to Example 9 | 5% |

A manner of preparation of the above face cream could consist in heating fraction A and fraction B separately at 75° C., adding fraction A to fraction B with turbine agitation, then cooling the mixture A and B to 40° C. and introducing fraction C into it, then cooling the whole to ambient temperature.

### EXAMPLE 15

Cosmetic product in the form of a massage gel based on exfoliant microparticles freeing an active thinning principle upon application

| | | |
|---|---|---|
| A | Dimethicone and Cyclomethicone and | 5.00% |
| | Dimethiconol (Abil OSW 12) | |
| B | Water | 84.90% |
| | Preservative | 0.40% |
| | Glycerine | 4.00% |
| | Xanthane gum | 0.20% |
| | Sepigel 305 | 2.50% |
| C | Microparticles according to Example 7 | 3% |

A mode of preparation of the message gel above could consist in dispersing fraction A and fraction B with turbine agitation, and adding to it fraction C with moderate agitation.

Of course, the invention is not limited to the described embodiments. Modifications remain possible, particularly as to the construction of various elements or by substitution of technical equivalents, without thereby departing from the scope of protection of the invention.

What is claimed:

1. A matrix for forming microparticles or nanoparticles, comprising:
at least one hydrophobic body solid at ambient temperatures wherein the solid hydrophobic body, or a mixture of hydrophobic solid bodies constitute from 30% to 60% by weight of the matrix,
at least one liquid body liquid at ambient temperatures, or a mixture of liquid hydrophobic bodies and liquid hydrophilic bodies present in an amount of 40% to 70%, and
wherein said liquid body is totally or partially replaced with an active principal so as to form a nanoparticle or microparticle heavily loaded with said active principal,
wherein said nanoparticles and microparticles are formed by heating, mixing and solubilizing the solid body or bodies in the liquid body or bodies, said matrix having a melting temperature comprised between 25° C. and 85° C.

2. The matrix for forming microparticles or nanoparticles according to claim 1, wherein the solid body or the mixture of solid bodies, and the liquid body or the mixture of liquid bodies, are selected in proportions so that the melting temperature of the matrix is comprised between 50° C. and 75° C.

3. The matrix for forming microparticles or nanoparticles according to claim 1, wherein the solid hydrophobic body, or the mixture of hydrophobic solid bodies is selected from the group consisting of solid fatty bodies, waxes and hydrophobic polymers used in cosmetics.

4. The matrix for forming microparticles or nanoparticles according to claim 1, wherein a liquid hydrophobic body, or a mixture of liquid hydrophobic bodies is selected from hot solvents for the solid hydrophobic body or the mixture of bodies entering into the matrix, wherein the liquid hydrophobic body, or the mixture of liquid hydrophobic bodies is selected from liquid oils used in cosmetic compositions.

5. A process for producing microparticles or nanoparticles adapted to encapsulate lipophilic, hydrophobic and/or hydrophilic active substances, comprising:
preparing a matrix by solubilizing by heating at least one solid hydrophobic body or a mixture of solid hydrophobic bodies when at ambient temperature, in at least one liquid body or a mixture of liquid bodies, hydrophobic and/or hydrophilic, at a temperature higher than the melting temperature of the resulting matrix, wherein the liquid body, or a mixture of liquid bodies constitute from 40% to 70% by weight of the matrix and are selected from hot solvents for the solid hydrophobic body or bodies entering into the matrix,
wherein said liquid body is totally or partially replaced with an active principal so as to form a nanoparticle or microparticle heavily loaded with said active principal, and
dispersing the matrix hot, in liquid or substantially liquid form, in an immiscible gaseous or liquid medium, cold so as to obtain a spontaneous solidification of fine droplets of liquid dispersed in a form of microparticles or nanoparticles

6. The process according to claim 5, wherein the melting temperature of the resulting matrix is between 25° C. and 85° C.

7. The process according to claims 5, wherein the solid hydrophobic body, or a mixture of solid hydrophobic bodies constitutes from 30 to 60% by weight of the matrix and are selected from the group consisting of solid fatty bodies, waxes and hydrophobic polymers used in cosmetics.

8. The process according to claims 5, further comprising encapsulating the hydrophobic, lipophilic and/or hydrophilic active substance or substances in the matrix by dispersing or solubilizing the hydrophobic, lipophilic and/or hydrophilic active substance or substances in a liquid condition before cooling and dispersing the matrix.

9. The process according to claim 5, further comprising encapsulating a lipophilic active principle by adsorbing hydrophilic active substances present in an aqueous phase on the surface of the formed microparticles.

10. The process according to claim 9, further comprising producing positively or negatively charged microparticles or nanoparticles by selecting the natures and proportions of constituents of the matrix and of a dispersion medium in a suitable manner, adjusting the dispersion medium to a suitable pH, followed by complexing the microparticles or nanoparticles by one or several active principles of an opposite charge or having local opposite charges in its structure.

11. The process according to claim 10, further comprising isolating from the dispersion medium microparticulate aggregates formed by adsorption of charged hydrophilic active principles, by ionic complexing on microparticles or nanoparticles of opposite charge, followed by drying the microparticulate aggregates.

12. The process according to claim 5, further comprising dehydrating microparticles or nanoparticles in suspension to produce a final pulverulent product.

13. Microparticles or nanoparticles obtained by the process of production according to claim 13, wherein the microparticles or nanoparticles have sizes between 5 millimeters and 10 nanometers.

14. The microparticles or nanoparticles according to claim 13, wherein the microparticles or nanoparticles contain materials selected from the group consisting of liposoluble or lipodispersable active principles, liposoluble colorants, hydrophilic active principles in dispersed form, organic filers, mineral fillers, pigments, nacre-imparting agents and talc, the materials are maintained in an oily suspension with stabilizing additives selected from the group consisting of bentones, and silica.

15. The microparticles or nanoparticles according to claim 13, wherein the microparticles or nanoparticles carry one or more hydrophobic active principles selected from the group consisting of liposoluble vitamins, acetate, linoleate, tocopherol nicotinate, retinal palmitate, vitamin A propionate, ceramids, sterols, α bisabolol, hydrosterols, lecithins, phospholipids, liposoluble colorants, pigments, solar filters, diethylhexyl paramethoxycinnamate, essential oils and perfumes.

16. The microparticles or nanoparticles according to claim 13, wherein the microparticles or nanoparticles carry hydrophilic active principles in adsorbed form, selected from the group consisting of dihydroxyacetone, salicilic acid, enzymes, proteins, amino acids, hydrosoluble solar filters, vitamin C and vitamin D.

17. A composition for caring for skin and/or nails, comprising an active ingredient or active ingredient vehicle, and microparticles and/or nanoparticles according to claim 13.

18. A composition according to claim 17, wherein the microparticles and/or nanoparticles are present in an amount of 0.1% to 20% by weight of the composition.
